(19) United States
(12) Patent Application Publication
(10) Pub. No.: US 2025/0041348 A1
(43) Pub. Date: Feb. 6, 2025

(54) EXTRACELLULAR VESICLE THERAPY TO TREAT ORTHOPEDIC INJURY

(71) Applicant: Wisconsin Alumni Research Foundation, Madison, WI (US)

(72) Inventors: Peiman Hematti, Madison, WI (US); Connie Chamberlain, Monona, WI (US); John Kink, Madison, WI (US); Ray Vanderby, Madison, WI (US); Brian Walczak, Verona, WI (US)

(21) Appl. No.: 18/792,104

(22) Filed: Aug. 1, 2024

Related U.S. Application Data
(60) Provisional application No. 63/517,440, filed on Aug. 3, 2023.

(51) Int. Cl. A61K 35/28; A61P 19/04; C12N 5/0775

(52) U.S. Cl. CPC A61K 35/28; A61P 19/04; C12N 5/0663; C12N 2501/25; C12N 2509/00

(57) ABSTRACT

An ex vivo generated population of tissue-specific extracellular exosomes and methods of making and using such exosomes for treating orthopedic injury are provided.

### CROSS-REFERENCE TO RELATED APPLICATION

This application claims priority to U.S. Provisional Application No. 63/517,440, filed Aug. 3, 2023, which is incorporated by reference in its entirety.

### BACKGROUND OF THE DISCLOSURE

### Field of the Disclosure

The present disclosure relates to treatment of orthopedic injuries using mesenchymal stromal cell (MSC)-derived extracellular vesicles, in a subject in need thereof.

### Technical Background

In the United States there are between 100,000 and 200,000 ligament ruptures per year. (Musahl V, Karlsson J. Anterior Cruciate Ligament Tear. N Engl J Med 2019; 380:2341) Each year there may be as many as 2.5 million Achilles tendon ruptures (Soslowski L) and the incidence of rotator cuff tears is even higher. (Yamamoto A, et al.) While most ligament and tendon injuries occur in athletes, almost anyone is susceptible to injury. Ligament and tendon healing after injury is a complex process that forms a neo-ligament, more scar-like than its native tissue. The repair process can extend for years, and the healed structure never fully recovers its mechanical properties. Despite surgical and physical therapy advancements to improve ligament and tendon healing, none eliminate scar formation, thereby making them prone to re-injury.

Extracellular vesicle therapy is a promising therapeutic modality for tendon and ligament injuries. Exosome-educated macrophages, administered in a mouse model of tendon injury have been shown to contribute to improved tendon healing by positively impacting mechanical strength and stiffness of healing tendons. However, there remains a need in the field for extracellular vesicle and cell-based therapies that promote ligament and tendon healing while improving mechanical properties of healing tendons and/or ligaments.

### SUMMARY OF THE DISCLOSURE

The disclosure describes methods and composition for improving the healing and mechanical properties of injured and/or surgically repaired orthopedic tissues, specifically tendons and ligaments.

In a first aspect, the present disclosure provides a method of treating an orthopedic injury in a subject in need thereof, the method includes administering to the subject a population of non-preconditioned mesenchymal stromal cell (MSC)-derived extracellular vesicles thereby improving a mechanical property of the orthopedic injury.

In one embodiment of the first aspect, the orthopedic injury is selected from the group consisting of a partial tendon tear, a complete tendon tear, a partial tendon laceration, a compete tendon laceration, a partial tendon avulsion, a complete tendon avulsion, a partial ligament tear, a complete ligament tear, a partial ligament laceration, a compete ligament laceration, tendinopathy, tendinosis, tendinitis, meniscal tears, and joint capsule tears. In one embodiment of the first aspect, the orthopedic injury is selected from the group consisting of plantar fasciitis, tennis elbow, bicep tendinitis, and carpal tunnel syndrome. In one embodiment of the first aspect, the orthopedic injury is a medial collateral ligament (MCL) injury. In one embodiment of the first aspect, the orthopedic injury is an anterior cruciate ligament (ACL) injury. In one embodiment of the first aspect, the population of MSC-derived extracellular vesicles is obtained by culturing a population of bone marrow mesenchymal stem cells (BM-MSCs) in vitro and isolating the population of MSC-derived extracellular vesicles from the BM-MSCs. In one embodiment of the first aspect, the population of MSC-derived extracellular vesicles (EVs) is administered at a dose between about 1×104 EVs/kilogram and about 10×109 EVs/kilogram of body weight of the subject. In one embodiment of the first aspect, the population of MSC-derived extracellular vesicles is administered by intravenous, intramuscular, subcutaneous, or intradermal injection or infusion. In one embodiment of the first aspect, the mechanical property is maximum load of a tendon and/or ligament. In one embodiment of the first aspect, the mechanical property is maximum stress of a tendon and/or ligament.

In a second aspect, the present disclosure provides a pharmaceutical composition including a population of non-preconditioned mesenchymal stromal cell (MSC)-derived extracellular vesicles and a pharmaceutically acceptable carrier.

In a third aspect, the present disclosure provides a method of improving mechanical properties of an orthopedic injury. The method includes the steps of a) administering to a subject in need thereof a population of non-preconditioned mesenchymal stromal cell (MSC)-derived extracellular vesicles to a site of an orthopedic injury, wherein the orthopedic injury comprises an injured tissue, wherein the injured tissue comprises a tendon, a ligament, cartilage, a peri-ligamentous tissue, a peri-tendinous tissue, and/or a joint capsule; and b) improving a mechanical property of the injured tissue.

In one embodiment of the third aspect, the mechanical property is maximum load of a tendon and/or ligament. In one embodiment of the third aspect, the mechanical property is maximum stress of a tendon and/or ligament.

In a fourth aspect, the present disclosure provides a method of improving scar healing and/or collagen organization in a subject with an orthopedic injury, comprising: a) preconditioning mesenchymal stromal cells (MSC); b) isolating extracellular vesicles from the preconditioned MSCs; and c) administering an effective amount of preconditioned MSC-derived extracellular vesicles systemically or locally to the subject.

In one embodiment of the fourth aspect, the population of MSC-derived extracellular vesicles is administered to the subject at a dose equal to, or less than about 1×10¹⁰ extracellular vesicles/kg of body weight.

In a fifth aspect, the present disclosure provides a method of obtaining extracellular vesicles from a population of preconditioned mesenchymal stromal cells (MSCs), comprising the steps of a) obtaining a population of MSCs from bone marrow of a subject; b) preconditioning the population of MSCs; and c) isolating the extracellular vesicles from the population of preconditioned MSCs.

In one embodiment of the fifth aspect, the preconditioning comprises treating the MSCs with an inflammatory factor. In one embodiment of the fifth aspect, the inflammatory factor comprises tumor necrosis factor (TNF), Interferon-gamma (IFN-γ), Interleukin-1 beta (IL-1B), Interleukin-6 (IL-6), Interleukin-17 (IL-17). In one embodiment of the fifth aspect, the inflammatory factor comprises polyinosinic acid, hypoxia, lipopolysaccharide (LPS), or an LPS derivative. In one embodiment of the fifth aspect, the preconditioning comprises treating the MSCs with an LPS analog. In one embodiment of the fifth aspect, the LPS analog is CRX, monophosphoryl lipid A (MPLA), lipid A, Eritoran (E5564), RC-529, CRX-526, GSK1795091, and/or SMIP-30. Additional LPS analogs include those disclosed in AG Stover (“Structure-activity relationship of synthetic toll-like receptor 4 agonists” J Biol Chem. 2004).

In one embodiment of any of the previous aspects or embodiments thereof, the extracellular vesicles include one or more exosomes, microvesicles, and/or apoptotic bodies.

In one embodiment of the second aspect, the extracellular vesicles comprise one or more exosomes, microvesicles, and/or apoptotic bodies.

These and other features and advantages of the present invention will be more fully understood from the following detailed description taken together with the accompanying claims. It is noted that the scope of the claims is defined by the recitations therein and not by the specific discussion of features and advantages set forth in the present description.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF DRAWINGS

The accompanying drawings are included to provide a further understanding of the methods and compositions of the disclosure and are incorporated in and constitute a part of this specification. The drawings illustrate one or more embodiment(s) of the disclosure, and together with the description serve to explain the principles and operation of the disclosure. Such detailed description refers to the following drawings.

FIG. 1. Overview of exosome isolation and treatment of orthopedic injury wherein bone marrow-derived mesenchymal stromal cells (BM-MSC) are collected from the Iliac crest or other sources and cultured in vitro followed by exosome isolation from cultured BM-MSCs. PBS Control (CX) or low dose isolated exosomes (EXO; 1×10⁹ exosome/leg) are administered at the time of medial collateral ligament (MCL) injury. Mechanical testing and immunohistochemistry staining of the MCL in CX and EXO treatment groups occurred at fourteen days post-MCL injury.

FIGS. 2A and 2B. (2A) Failure load (N) testing 14 days after MCL injury in PBS-Control (CX) or EXO treatment groups. No changes in MCL load testing were observed between CX and EXO-treatment groups (p=0.79). (2B) Maximum (Max) stress (MPa) testing 14 days after MCL injury in PBS-Control (CX) versus EXO treatment. No changes in MCL failure load were observed between CX and EXO treatment groups (p=0.97)

FIGS. 3A-3J. Immunohistochemistry and histology results of the day 14 post-injured medial collateral ligament (MCL) after treatment with PBS-Control (CX) or exosomes (EXO). (3A) M1 macrophage presence (Density/mm²) 14 days post MCL injury after EXO or PBS (CX) administration. No difference in the number of M1 macrophages was noted at 14 days post-injury (p=0.33). (3B) M1 macrophage staining 14-days post MCL injury in CX (left panel) versus EXO treated MCLs (right panel). (3C) M2 macrophage presence (Density/mm²) at 14 days post MCL injury after EXO versus PBS (CX) treatments. No difference in M2 macrophage density was present at day 14 (p=0.74). (3D) M2 macrophage staining within the injured MCL after treatment with PBS (CX; left panel) versus EXO (right panel). (3E) Collagen organization (global organization) 14 days post MCL injury comparing EXO treatment versus CX. EXO treatment improved collagen organization within the day 14 healing MCL versus the CX (p=0.01). (3F) H&E staining for scar size at 14 days post MCL injury with EXO treatment versus CX. The outlined regions represent the borders between organized with parallel collagen fibers (tendon-like) and poorly organized (scar-like) collagen fibers. EXO treatment (1×10⁹ exosomes) reduced scar size versus CX (outlined area; p=0.03). (3G) Type I collagen localization (density/mm²) within the wound at 14 days post MCL injury after EXO treatment versus CX. Type I collagen was increased at 14 days post MCL injury with exosome treatment versus CX (p=0.04). (3H) Immunohistochemistry of Type I collagen 14 days post MCL injury after EXO treatment (bottom panel) versus CX (top panel) (3I) Type III collagen (density/mm²) within the wound at 14 days post MCL injury with EXO treatment versus CX. Type III collagen density was increased at 14 days post MCL injury with EXO treatment versus CX (p=0.01). (3J) Representative examples of type III collagen immunohistochemistry within the day 14 post-injured MCL after exosome treatment (bottom panel) versus CX (top panel). Scale bars=50 μm.

FIG. 4. Overview of technique to isolate exosomes from pre-conditioned MSCs for use in high-dose exosome in vivo studies. Mesenchymal stromal cells (MSCs) were isolated from the iliac crest and expanded in standard cell culture media (serving as the control) or expanded in culture media preconditioned with 100 ng/mL TNF-α (human, recombinant SRP3177 from Sigma-Aldrich; TNF-EXO), or 100 ng/mL CRX-527 (an LPS analog, Cat. No. tlrl-crx527 from In VivoGen; CRX-EXO). MSC-derived exosomes were isolated via ultracentrifugation and used as treatments using a rat MCL injury model. Rats underwent bilateral MCL transection immediately followed by administration of PBS (serving as control; CX), 5×10⁹ exosomes without preconditioning (EXO), 5×10⁹ exosomes with TNF preconditioning (TNF-EXO), or 5×10⁹ exosomes with CRX preconditioning (CRX-EXO). The contralateral MCL was administered a second dose of the same treatment 3 days post injury (when macrophage presence is elevated). Ligaments were collected at days 14 post-injury and healing was assessed via mechanical testing. Data were analyzed via Student's T-test (treatment vs control).

FIGS. 5A-5D. Mechanical testing results of the healing ligament fourteen days post MCL-injury. (5A) Maximum (Max) load (N) 14 days after injury in PBS (CX), EXO, CRX-EXO, or TNF-EXO groups. High dose exosome administration without preconditioning (EXO; 5×10⁹ exosomes) at time of injury improved the maximum load of the MCL at 14-days post injury compared to CX, CRX-EXO and TNF-EXO groups (p<0.05). 5B) Maximum (Max) stress (MPa) 14 days after injury in CX, EXO, CRX-EXO, and TNF-EXO groups. High dose EXO administration at time of injury improved the maximum stress of the MCL at 14-days post injury compared to CRX-EXO groups (p=0.003). (5C) Stiffness (N/mm) 14 days after injury in CX, EXO, CRX-EXO, or TNF-EXO groups. High dose EXO administration at time of injury improved the maximum stiffness of the MCL at 14-days post injury compared to CRX-EXO, and TNF-EXO groups (p<0.05). (5D) Modulus (MPa) 14 days after injury in CX, EXO, CRX-EXO, or TNF-EXO groups. High dose EXO administration at time of injury improved modulus of the MCL at 14-days post injury compared to CRX-EXO (p=0.013).

FIGS. 6A-6D. Mechanical testing results of the MCL fourteen days post-injury to examine day effect of exosome administration. (6A) Maximum (Max) load (N) 14 days after injury in CX, EXO, CRX-EXO, or TNF-EXO groups receiving exosome treatment immediately following MCL injury (Day 0) or at day 3 post-injury. A second dose of PBS (CX), EXO, CRX-EXO, or TNF-EXO at day 3 post injury had no additional beneficial effect on maximum load compared to a single dose. (6B) Maximum (Max) stress (MPa) 14 days after injury in CX, EXO, CRX-EXO, or TNF-EXO groups receiving a single administration immediately following MCL injury (left bar of each group) or at day 3 following MCL injury (right bar of each group). A second dose of PBS (CX), EXO, CRX-EXO, or TNF-EXO delivered 3 days post injury had no additional effect on maximum stress of the MCL 14 days after injury in any group when compared to a single dose. (6C) Stiffness (N/mm) 14 days after injury in CX, EXO, CRX-EXO, or TNF-EXO groups receiving a single administration immediately following MCL injury (left bar of each group) or at day 3 following MCL injury (right bar of each group). A second dose of PBS (CX), EXO, CRX-EXO, or TNF-EXO at Day 3 post injury had no additional beneficial effect on stiffness of the MCL 14 days after injury in any group when compared to a single dose. (6D) Modulus (MPa) 14 days after injury in CX, EXO, CRX-EXO, or TNF-EXO groups receiving a single administration immediately following MCL injury (left bar of each group) at day 0 and again at day 3 following MCL injury (right bar of each group). A second dose of PBS (CX), EXO, CRX-EXO, or TNF-EXO at day 3 post injury had no additional beneficial effect on modulus of the MCL 14 days after injury in any group when compared to a single dose.

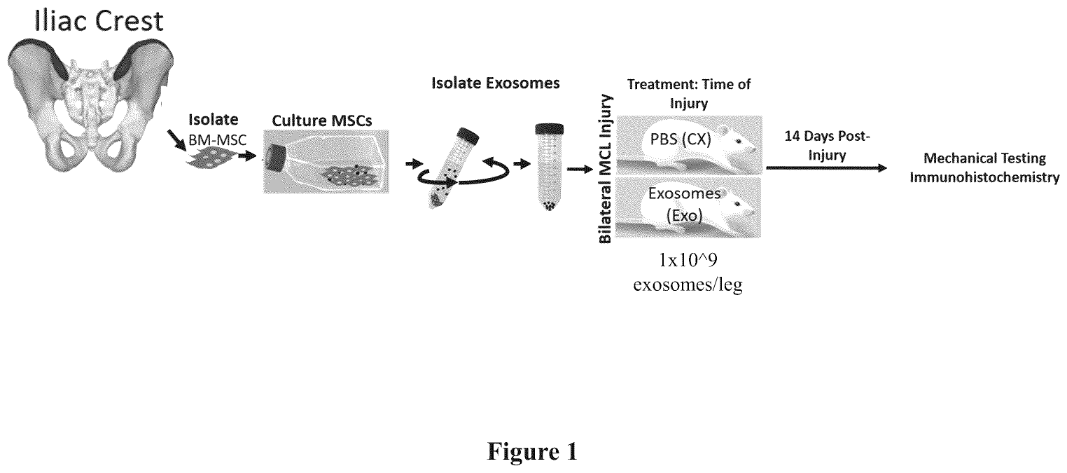

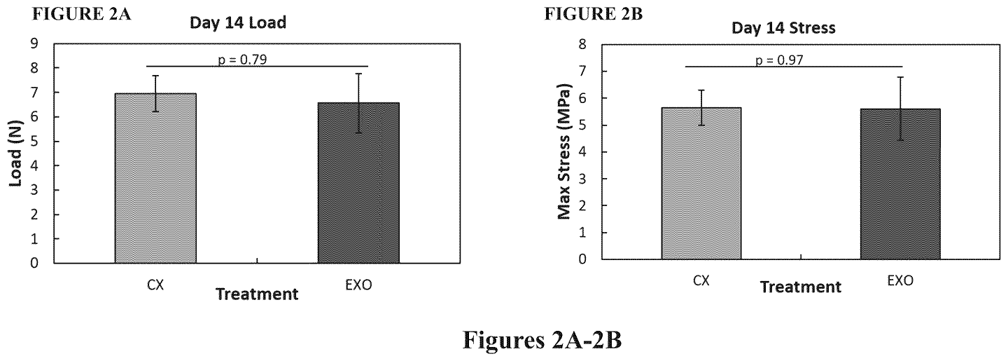

### DETAILED DESCRIPTION

Provided herein are methods and compositions for treatment of orthopedic injuries and/or healing of tissue following orthopedic surgery, such as tendon and ligament repair.

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” may be replaced with either of the other two terms, while retaining their ordinary meanings

As used herein, the singular forms “a,” “an,” and “the” include plural referents unless the context clearly indicates otherwise.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

The terms “subject” and “patient” can be used interchangeably and refer to a warm-blooded animal such as a mammal, preferably a human, which is afflicted with, or has the potential to be afflicted with an orthopedic injury as described herein.

The phrase “in need thereof” indicates the state of the subject, wherein therapeutic or preventative measures are desirable. Such a state can include, but is not limited to, subjects having a condition or injury as described herein or a pathological symptom or feature associated with a condition or injury as described herein. Subjects in need of treatment can also include those suspected of having a condition or injury as described herein. Treating a subject suspected of having a condition or injury described herein further includes initiating the administration of a therapeutic modality, for example extracellular vesicles as described herein, before the injury progresses in severity.

The terms “prevent” and “preventing” refer to prophylactic or preventive measures intended to inhibit undesirable physiological changes or detrimental progression of an injury.

### Compositions

“Pharmaceutical composition” as used herein refers to a composition that includes one or more therapeutic agents, such as an extracellular vesicle or preconditioned extracellular vesicle, a pharmaceutically acceptable carrier, a solvent, an adjuvant, and/or a diluent, or any combination thereof. In some embodiments, pharmaceutical compositions contemplated herein include extracellular vesicle in high doses (1×10⁹ extracellular vesicles). The exact nature of the carrier, solvent, adjuvant, or diluent will depend upon the desired use of the composition (e.g., route of administration), and may range from being suitable or acceptable for veterinary uses to being suitable or acceptable for human use.

“High Dose” as used herein refers to an extracellular vesicle dose that is about 3, about 4, about 5, about 6, about 7, about 8, about 9, or about 10 times greater than the extracellular vesicle dose that does not improve indices of ligament or tendon healing, as measured by mechanical testing.

Examples of compositions appropriate for such therapeutic applications include preparations for parenteral, subcutaneous, transdermal, intradermal, intramuscular, intravenous (e.g., injectable), intrajoint, intratendon, intraligament, intrasynovial, extrasynovial, or intratracheal administration, such as sterile suspensions, emulsions, and aerosols. In some cases, pharmaceutical compositions appropriate for therapeutic applications may be in admixture with one or more pharmaceutically acceptable excipients, diluents, or carriers such as sterile water, physiological saline, glucose, or the like. For example, extracellular vesicles described herein can be administered to a subject as a pharmaceutical composition comprising a carrier solution. In some cases, pharmaceutical compositions are lyophilized. In other cases, pharmaceutical compositions as provided herein contain auxiliary substances such as wetting or emulsifying agents, pH buffering agents, gelling or viscosity enhancing additives, preservatives, flavoring agents, colors, and the like, depending upon the route of administration and the preparation desired. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

As used herein, the term “exosomes” (30-200 nm) refers to a subgroup of EVs small lipid vesicles released by a variety of cell types. Exosomes are generated by inward- or reverse budding, resulting in particles that contain cytosol and exposed extracellular domains of certain membrane-associated proteins (Stoorvogel et al., Traffic 3:321-330 (2002)). Methods of preparing exosomes from cells are known in the art. See, for example, Raposo et al., J. Exp. Med. 183:1161 (1996). In one method, exosomes are recovered from conditioned culture medium by centrifugation.

“Extracellular vesicles” or EVs, as used herein refers to a heterogeneous population of lipid bound vesicles secreted by a variety of cells into the extracellular space. The vesicles originate in the endosomal system or can be shed from the plasma membrane and are released from a wide variety of cells. Extracellular vesicles are generally thought to be unable to replicate and contain lipids, nucleic acids, and proteins. Extracellular vesicles, as used herein, includes small (<200 nm) and medium/large EVs (>200 nm), those with low, middle, or high density, and EVs that are cell or condition specific. In an exemplary embodiment, EVs include exosomes (30-200 nm), microvesicles (50-1000 nm), and apoptotic bodies (500-2000 nm). Without wishing to be bound by theory, it is believed that all 3 groups of EVs may contribute to the therapeutic effects described herein. Extracellular vesicles have been shown to be involved in physiological, pathological, and immune processes and have been identified as mechanism of intercellular communication via exchange of vesicle content.

Exosomes or intraluminal vesicles (ILVs) are small (about 10-200 nm) lipid membrane-bound vesicles that participate in cell-to-cell communication in almost all cell types by transferring specific host cell derived nucleic acid/protein to targeted recipient cells and reprogramming cell behavior. Without wishing to be bound by any particular theory, it is understood that tissue specific exosomes express surface markers or contain DNA, RNA, microRNA, or proteins specific to their tissue of origin. Exosomes from a tissue of interest, for example, a damaged tissue targeted for repair, are likely to contain tissue-specific translational or post-translational factors, internal nucleic acids, and proteins specific to the tissue of interest and superior for repair of said tissue. Exosomes may be isolated or derived from any cell type that resides in the target tissue of interest which can be isolated and cultured for a period of time appropriate for the isolation of exosomes. In some embodiments, extracellular vesicles are isolated from cultured MSC. Exosomes suited for use in the methods can be derived fresh or can be previously frozen and thawed. In some embodiments, exosome preparations may also include micro-vesicles. Exosomes can have, but are not limited to, a diameter of about 10-300 nm. In some embodiments, the exosomes can have, but are not limited to, a diameter between 20-250 nm, 30-200 nm, or about 50-150 nm.

In some embodiments, as used herein, extracellular vesicles can refer to a population of lipid bound vesicles made up primarily (e.g., greater that about 50%, 60%, 70%, 80%, or 90%) of exosomes.

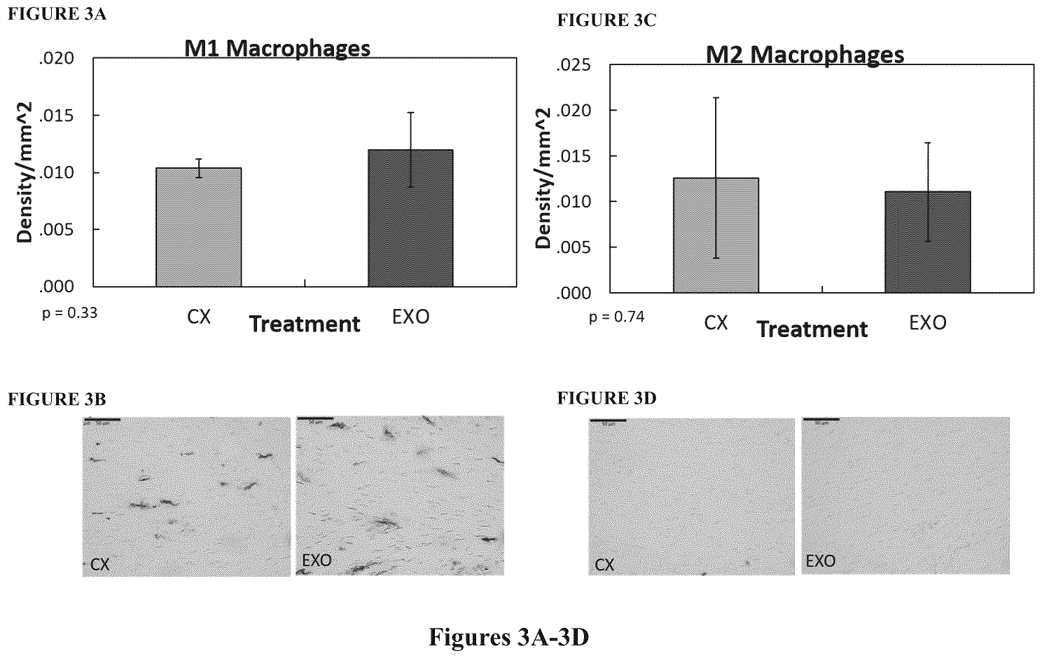

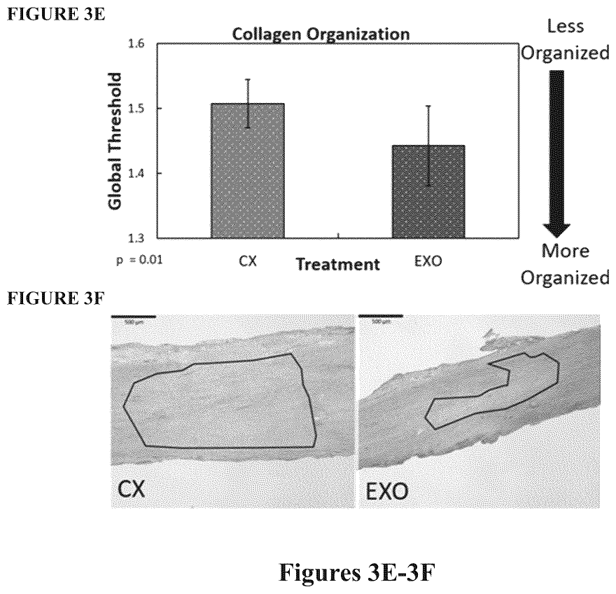

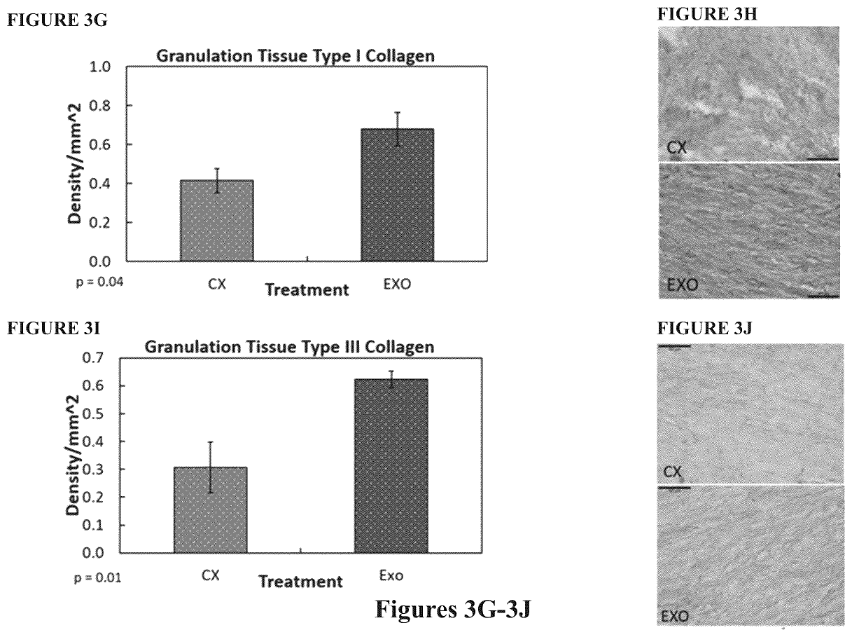

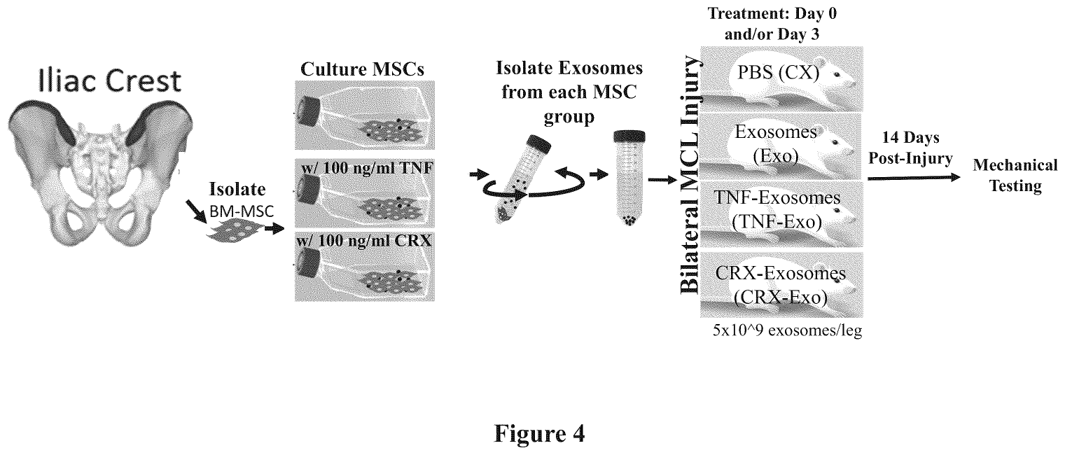

Methods of preparing extracellular vesicles from cells are known in the art. See, for example, Raposo et al., J. Exp. Med. 183:1161 (1996). In some embodiments, extracellular vesicles are recovered from cell culture medium by centrifugation. In previous work the inventors have demonstrated that administration of extracellular vesicles (1×10⁹) directly into the MCL at the time of injury improved ligament remodeling as assessed by reduced MCL scar size, improved collagen organization, and increased Type I and Type III collagen production within the ligament (FIGS. 3E-3J). However, direct extracellular vesicle injection had no effect on mechanical properties of the ligament or reductions in inflammation of the MCL (FIGS. 3A-3D).

MSCs preconditioned with TNF-α and delivered to injured Achilles tendon, accelerate healing by improving mechanical function and reducing inflammation (Atkas et al., 2017). Healing effects were greater than treatments with unstimulated MSCs (Aktas, et al., 2017) or no treatment controls. Additional work has demonstrated that exosomes from lipopolysaccharide (LPS)-preconditioned MSCs prolong survival in a xenogeneic lethal acute radiation syndrome (ARS) mouse model (Kink et al., 2019).

Microvesicles, also referred to as ectosomes or microparticles, are extracellular vesicles that form by direct outward budding of the plasma membrane. Microvesicles are typically 100 nanometers to 1 micrometer in diameter.

Apoptotic bodies are released by dying cells into the extracellular space. Apoptotic bodies form by a separation of the plasma membrane from the cytoskeleton as a result of increased hydrostatic pressure after the cell contracts. Apoptotic bodies range in size from 50 nanometers to 5000 nanometers and contain intact organelles, chromatin, and small amounts of glycosylated proteins.

“Preconditioned exosomes” as used herein refers to exosomes derived from MSCs cultured in the presence of an additional bioactive agent. In one embodiment, tumor necrosis factor-alpha, TNF or TNF-α, (human recombinant, animal component-free, SRP3177) or CRX-527 (titl-crx527), a synthetic lipid A mimetic which binds and activates the Toll-like receptor 4 (TLR4), is added to cell culture as the preconditioning factor. Additional preconditioning or priming agents contemplated herein include bioactive molecules from prokaryotes/eukaryotes, including, cell membrane components, cytokines, chemokines, agonists, and adjuvants. In one embodiment, MSCs are harvested from iliac crest bone marrow. In some embodiments, MSC-derived exosomes are derived from peripheral blood, umbilical cord, placenta, tendons, adipose tissue, joints, synovial fluid, embryonic stem cells or induced pluripotent cells. The MSCs can be autologous or non-autologous. In some embodiments, the MSCs undergo preconditioning before exosome isolation to generate exosomes with different properties, producing different therapeutic effects with certain pretreatments. In some embodiments, preconditioning of MSCs comprises treating the MSCs with bioactive agents or other factors and harvesting the exosomes from the preconditioned-MSCs.

A variety of dosage schedules are contemplated by the present disclosure. For example, a subject can be dosed monthly, every other week, weekly, daily, or multiple times per day. Dosing can be done alone or at the time of surgery or other interventions. Dosage amounts and dosing frequency can vary based on the dosage form and/or route of administration, and the age, weight, sex, and/or severity of the subject's disease. In some embodiments of the present disclosure, the extracellular vesicles are administered via injection into the injury or into the area bordering the orthopedic injury.

The “therapeutic agents”, “therapeutic compounds”, or “compounds” described herein or compositions thereof, will generally be used in an amount effective to achieve the intended result, for example, in an amount effective to provide a therapeutic benefit to subject having an orthopedic injury or recovering from orthopedic surgery.

“Therapeutic benefit” refers to the rebuilding and/or remodeling of damaged tissue thereby eradicating or ameliorating one or more of the symptoms associated with the injury or surgical recovery such that a subject being treated with the therapeutic agent reports an improvement in feeling or condition, notwithstanding that the subject may still have incomplete healing or injury resolution.

Dosage amounts of the therapeutic agents and secondary therapeutic agents can be in the range of 10×10⁷ to 10×10¹² EVs/kilogram of body weight of the subject, but may be higher or lower, depending upon, among other factors, the activity of the extracellular vesicles, the bioavailability of the compounds within the extracellular vesicles, variations in metabolism kinetics, and other pharmacokinetic properties, the mode of administration and various other factors, including the particular injury being treated or surgery, the severity of existing or anticipated physiological dysfunction, the severity of the injury, the genetic profile, age, health, sex, diet, and/or weight of the subject. Dosage amounts and dosing intervals can be adjusted individually to maintain a desired therapeutic effect over time. For example, the compounds may be administered once, or once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### Methods

In some embodiments described herein is a method of treating an orthopedic injury in a subject in need thereof, the method comprising administering to the subject a population of non-preconditioned or preconditioned mesenchymal stromal cell (MSC)-derived extracellular vesicles thereby improving a mechanical property of the orthopedic injury. Subjects in need of treatment include those diagnosed with an orthopedic disease or injury or those who are recovering from an orthopedic-based surgery.

An “orthopedic condition” or “orthopedic injury” refers to, but is not limited to, conditions associated with an orthopedic or traumatic injury. Orthopedic injury may refer to, but is not limited to, partial or complete tendon or ligament tears, partial or complete tendon or ligament lacerations, partial or complete tendon or ligament avulsions, tendinopathy, tendinosis, tendinitis, meniscal tears, and joint capsule tears. Additional conditions associated with orthopedic injury include, but are not limited to arthritis, including osteoarthritis and rheumatoid arthritis, bursitis, plantar fasciitis, lateral epicondylitis (tennis elbow), medial epicondylitis (golf or baseball elbow), bicep tendinitis, carpal tunnel syndrome, repetitive motion injuries (e.g., iliotibial band), non-surgically repaired ligament injuries, non-surgically repaired tendon injuries. The tendon or ligament at risk of or affected by the orthopedic injury may be any tendon or ligament known in the body of a given subject. Conditions or injuries associated with orthopedic injury may affect, but are not limited to, injuries of the Achilles tendon, rotator cuff tendons (supraspinatus, infraspinatus, subscapularis, teres minor), biceps tendon, hip abductor and/or adductor tendons, anterior cruciate ligament, posterior cruciate ligament, medial collateral ligament, lateral collateral ligament, flexor tendons, extensor tendons, knee meniscus, shoulder labrum, hip labrum, joint capsule, patellar tendon, hamstring tendons, retinacula (e.g. flexor retinacula creating carpal tunnel), aponeuroses, tendon/ligament entheses, and fibrocartilage tissues including meniscus and labrums and hyaline cartilage tissues including articular cartilage.

“Mesenchymal stromal cells” (MSC) also referred to as “mesenchymal stem cells”, “marrow stromal cells”, or “multipotent stromal cells” refer to fibroblast-like cells that reside in virtually all postnatal tissues. MSC are multipotent, can be expanded and manipulated ex vivo and demonstrate the biological characteristics of differentiation and secretion of trophic factors and immunoregulatory properties. These characteristics allow for use of MSC in cellular therapy, generating new tissue, repairing injured tissue, including cartilage and more particularly tendons and ligaments. An MSC within the scope of this disclosure is any cell that can differentiate into osteoblasts, chondrocytes, tenocytes, myoblasts, astrocytes, and adipocytes. In some embodiments the MSC are isolated from the bone marrow. In one embodiment the MSC are isolated from the iliac crest of the pelvic girdle. MSC can also be isolated from blood, cartilaginous, skeletal, and fatty tissues.

An MSC within the scope of this disclosure is typically positive for the expression of CD105, CD73, and CD90 while lacking expression of CD45, CD34, CD14 or CD11b, CD79a or CD19, and HLA-DR surface molecules. (See Dominici et al. Minimal criteria for defining multipotent mesenchymal stromal cells. The International Society for Cellular Therapy position statement, (2006), Cytotherapy, 8 (4): 315-317). While these markers are known to characterize MSCs derived from most tissues, it is understood in the art that MSCs from some sources could exhibit differences in cell surface marker expression. In some embodiments, the tissue-specific cells are bone marrow mesenchymal stem cells (referred to herein as BM-MSCs). BM-MSCs can be cultured to generate extracellular vesicles (referred to herein as MSC-derived extracellular vesicles).

MSCs, BM-MSCs, tendon-MSCs, adipose-MSCs and other cells described herein for use in the methods or compositions of the present disclosure may be derived or isolated from any suitable source. In one embodiment, MSCs are isolated from tissue such as bone marrow, ligament, adipose, and tendon tissue. In one embodiment, MSCs are differentiated from embryonic or induced pluripotent stem cells.

MSCs, monocytes, macrophages, tissue-specific cells, and extracellular factors disclosed herein can be cultured or co-cultured in any medium that supports their survival and growth. In some embodiments, the medium is cell growth medium such as alpha MEM or RPMI-1640 with bovine serum or human serum depleted of extracellular vesicles. In some embodiments, the medium is serum free-medium including, but not limited to, X-VIVO™ 15 and STEMPRO™ serum-free media. In one embodiment, the medium uses human platelet lysates to replace the human AB serum in the MSC medium. In some embodiments, the culture can include secondary factors such as inflammatory factors such as cytokines, chemokines, or other immune modulator cells. Tissue-specific cells, extracellular factors, and macrophages can be autologous, syngeneic, allogeneic, xenogeneic, or third party with respect to one another.

In any of the methods of the present disclosure, the donor, and the recipient of the MSC-derived extracellular vesicles can be a single individual or different individuals, for example, autologous, allogeneic, or xenogeneic individuals. “Allogeneic” refers to something that is genetically different although belonging to or obtained from the same species (e.g., allogeneic tissue grafts or organ transplants). “Xenogeneic” refers to cells derived from a different species.

As used herein, the term “inflammatory” means relating to inflammation. The term “inflammation” refers to the process by which vascular tissues responds to harmful stimuli, such as pathogens, damaged cells, chemical or physical injuries, irritants or age-related degenerative processes. Inflammation includes, but is not limited to, secretion of and response to inflammatory factors, such as inflammatory cytokines. Inflammatory cytokines include, but are not limited to IL-1β, IFN-γ, and TNF-α.

As used herein, the terms “treat” and “treating” refer to both therapeutic and prophylactic or preventive measures, wherein the object is to prevent or slow (lessen) progression of an undesired physiological change or pathological disorder resulting from a disease or injury. For purposes of this disclosure, treating the disease or injury includes, without limitation, alleviating one or more clinical indications, decreasing inflammation, reducing the severity of one or more clinical indications of the disease or injury, diminishing the extent of the condition, stabilizing the condition or injury to prevent further deterioration, delaying or slowing, halting, or reversing the condition or injury and bringing about partial or complete elimination of the condition or injury. Treating can also refer to improvements in healing, strength, composition, wound size, function, adhesions, range-of-motion, or use of injured tissues(s). Treating the condition or injury also includes reducing the recovery time required before return to activity by days, weeks, months, or years as compared to prognosis if treated according to standard medical practice not incorporating treatment with extracellular vesicle.

The preferred route of administration may vary with, for example, the condition, injury, weight or the response to therapy. The formulations can also be administered by two or more routes, where the delivery methods are essentially simultaneous, or they may be essentially sequential with little or no temporal overlap in the times at which the compositions are administered to the subject. Suitable regimes for initial administration and further doses or for sequential administrations also are variable, may include an initial administration followed by subsequent administrations, but nonetheless, may be ascertained by the skilled artisan from this disclosure, documents cited herein, and the knowledge in the art.

In some cases, MSC-derived extracellular vesicles may be optionally administered in combination with one or more additional active agents or derived from MSCs preconditioned with one or more such active agents. Such active agents include anti-inflammatory, anti-cytokine, analgesic, antipyretic, antibiotic, and antiviral agents, as well as growth factors and agonists, antagonists, and modulators of immunoregulatory agents (e.g., TNF-α, IL-2, IL-4, TGFβ, IL-6, IL-10, IL-12, IL-13, IL-17, IL-18, IFN-α, IFN-γ, BAFF, CXCL13, IP-10, VEGF, EPO, EGF, HRG, Hepatocyte Growth Factor (HGF), Hepcidin, including antibodies reactive against any of the foregoing, and antibodies reactive against any of their receptors). Any suitable combination of such active agents is also contemplated. When administered in combination with one or more active agents, extracellular vesicles can be administered either simultaneously or sequentially with other active agents. For example, following orthopedic injury extracellular vesicles and a steroid or corticosteroid can be simultaneously administered for a length of time or according to a dosage regimen sufficient to support recovery and to treat, alleviate, or lessen the severity of the orthopedic injury.

In some embodiments, MSC-derived extracellular vesicles of the present disclosure may also be administered to a patient simultaneously undergoing surgical or endoscopic repair of the orthopedic injury. In such cases, extracellular vesicles can be provided to a subject in need thereof in a pharmaceutical composition adapted for direct administration to the tendon or ligament. Administration may be provided before, after or simultaneous with repair of the tendon or ligament. Surgery of ACLs, for example, is typically delayed allowing initial inflammation to subside. Without being bound by any particular theory, administration of MSC-derived extracellular vesicles prior to surgery could speed the process of reducing inflammation, shorten the time between injury and surgical treatment, initiate healing, and reduce injury-induced joint inflammation (associated with increased osteoarthritis in injured joints). Post-surgery administration of MSC-derived extracellular vesicles would reduce surgically induced complications associated with inflammation, angiogenesis, fibrosis, and degradation of repaired or replaced tissues as well as peri-ligamentous or peri-tendinous tissues (e.g., cartilage). MSC-derived extracellular vesicles can be administered directly to the tendon or ligament being repaired. MSC-derived extracellular vesicles may also be administered as part of a treatment in which the subject is receiving donor or graft tissues. MSC-derived extracellular vesicles may be applied via a collagen sponge or gel, hydrogel, or tissue engineered scaffold. Administration as part of a cell sheet or sheath around the tendon or ligament being treated is also envisioned.

In some embodiments, MSC-derived extracellular vesicles are administered to a subject in need thereof using an infusion, topical application, surgical transplantation, or implantation. In an exemplary embodiment, administration is systemic. In such cases, MSC-derived extracellular vesicles can be provided to a subject in need thereof in a pharmaceutical composition adapted for parenteral, subcutaneous, transdermal, intradermal, intramuscular, intravenous (e.g., injectable), intrajoint, intratendon, intraligament, intrasynovial, extrasynovial, or intratracheal administration, such as in sterile suspensions, emulsions, and aerosols.

In one embodiments, extracellular vesicles are administered using intravenous injection. Typically, compositions for intravenous administration are solutions in sterile isotonic aqueous buffer. The use of such buffers and diluents is well known in the art. Where necessary, the composition may also include a local anesthetic to ameliorate any pain at the site of the injection. Generally, the ingredients are supplied either separately or mixed together in unit dosage form. In one embodiment the components are supplied as a cryopreserved concentrate in a hermetically sealed container such as an ampoule indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration. In some cases, compositions comprising extracellular vesicles are cryopreserved prior to administration. In one embodiment, the compositions for intravenous or other forms of administration contemplated herein are packaged as a kit that also includes needles, syringes, and instructions for use.

A “therapeutically effective dose” or “therapeutically effective amount” is a quantity of extracellular vesicles sufficient to affect a beneficial or desired clinical result. The effective dose can be administered in one or more doses at one or more time points. Therapeutically effective amounts for administration to a human subject can be determined in animal tests and any art-accepted methods for scaling to determine an amount effective for human administration from an amount effective in an animal test by using conversion factors known in the art. The effective amount obtained in one animal model can also be converted for another animal model by using suitable conversion factors such as, for example, body surface area factors.

In some cases, an effective dose of MSC-derived extracellular vesicles (EVs) is about 1×10⁴ EVs/kilogram to about 10×10¹¹ EVs/kilogram of body weight of the recipient. Effective amounts will be affected by various factors which modify the action of the cells upon administration and the subject's biological response to the cells, e.g., severity of the orthopedic injury, type of damaged tissue, the patient's age, sex, and diet, the severity of inflammation, time of administration, route of administration, and other clinical factors. In some embodiments, the effective dose is about 1×10⁷ EVs/kilogram to about 6×10⁷ EVs/kilogram. In one embodiment, the effective dose is about 5×10⁷ EVs/kilogram. In some embodiments, the effective dose is 1×10⁴ EVs/kilogram to about 10×10⁹ EVs/kilogram based on the estimated weight of the tendon or ligament being treated.

It is to be understood that, for any particular subject, specific dosage regimes should be adjusted over time according to the individual need and the professional judgment of the person administering or supervising the administration of the extracellular vesicle. For example, an MSC-derived extracellular vesicle dosage for a particular subject with an orthopedic injury can be increased if the lower dose does not elicit a detectable or sufficient improvement in the orthopedic injury. Conversely, the dosage can be decreased if the orthopedic injury is treated or eliminated.

In some cases, therapeutically effective amounts of MSC-derived extracellular vesicles can be determined by, for example, measuring the effects of a therapeutic in a subject by incrementally increasing the dosage until the desired symptomatic relief level is achieved. A continuing or repeated dose regimen can also be used to achieve or maintain the desired result. Any other techniques known in the art can be used as well in determining the effective amount range. The specific effective amount will vary with such factors as the particular disease state being treated, the physical condition of the subject, the type of animal being treated, the duration of the treatment, and the nature of any concurrent therapy.

### EXAMPLES

The Examples that follow are illustrative of specific embodiments of the disclosure, and various uses thereof. They are set forth for explanatory purposes only and should not be construed as limiting the scope of the disclosure in any way.

### Example: Extracellular Vesicle Therapy and Ligament Healing after Injury

The ability of extracellular vesicles to stimulate medial collateral ligament (MCL) injury healing was determined by administering normal and preconditioned MSC-derived exosomes directly into the MCL at the time of injury in a rat model. All animals received exosomes at the time of MCL injury with a population of animals also receiving a second administration of exosomes at three days post injury.

### Methods

All protocols were approved by the Health Sciences Institutional Review Board of University of Wisconsin-Madison School of Medicine and Public Health.

### MSC Collection and Cell Culture

Mesenchymal stem cells (MSCs) were isolated from filters left over after bone marrow (BM) harvest from normal healthy donors to human leukocyte antigen (HLA)-matched siblings or from human donor hamstring or bicep tendon tissue.

BM-MSCs, were recovered by rinsing the filter with phosphate-buffered saline (PBS) followed by separation of mononuclear cells using FICOLL™-Hypaque 1.073 (GE Healthcare Biosciences). Red blood cells were lysed with a 3-minute incubation in ACK lysis buffer and mononuclear cells were suspended in α-minimum essential medium supplemented with 10% fetal bovine serum (US origin, uncharacterized; Hyclone, Logan, UT, USA), nonessential amino acids (1×), and 4 mM L-glutamine. Cells were cultivated in 75-cm² filter cap cell culture flasks.

MSCs were also isolated from resected hamstring tendon or bicep tendon samples by mincing samples in MSC culture media containing 0.5% w/v of collagenase type 1 (from C. histolyticum). The sample was digested for about 18 hours at 37° C., 5% CO₂, the digested material was passed through a 100 μM cell strainer to remove clumps, and the flow-through collected and centrifuged at 500×g for 10 minutes. The cell pellet was re-suspended in MSC culture medium and incubated for 18 hours allowing for MSC attachment.

Attached BM and tendon MSCs (passage 0) were harvested using TrypLET cell dissociation enzyme (Invitrogen) and re-plated into new flasks (Kim J et al., Exp Hematology (2009)). Each subsequent re-plating was considered a separate passage, and cells were verified as MSCs by flow cytometry (Dominici M et al., Cytotherapy 8 (4): 315-317 (2006)). After confirmation, BM-MSC isolates were used for exosome isolation.

Exosome Isolation from MSC

Human MSCs derived from BM or tendons were grown to confluence using MSC-culture medium in 75-cm² filter cap cell culture flasks. The culture media were removed from adherent MSCs washed with PBS, and serum containing culture medium was replaced with StemPro™ MSC serum-free medium (SFM) CTS (A103332-01, Gibco Life Technologies), which is largely free-of serum-based endogenous exosomes. MSCs were primed or stimulated with either PBS as non-preconditioned controls (CX) or conditioned with 1) 100 ng/ml of TNF-α, or 2) 100 ng/ml of the lipid A analog CRX-527 for 18-24 hours. The conditioned culture medium (CM) was collected, and exosomes were isolated by a two-step centrifugation process; a low-speed centrifugation of 2000×g for 20 minutes followed by a high-speed ultracentrifugation of 100,000×g for 2 hours. (Thery C. et al., Current Protocols in Cell Biology (2006)). Exosome pellets were resuspended in PBS, and exosome particle size distribution (mean and mode size, nm) and particle number per/mL were determined using nanoparticle tracking analysis using IZON qNano Nanoparticle, Nanosight NS300 or Zetaview instruments. Multiple cycles of exosome isolation can be performed on the non-conditioned control MSC exosomes and CRX-exosomes.

### Animal Model of MCL Injury

All procedures were approved by the University of Wisconsin Institutional Animal Use and Care Committee. The MCL was surgically transected in five-week-old male Foxn1^{-/-} (nude) rats or male Wistar rats (250-400 g) to create a uniform defect for determining exosome mediated MCL healing. Rats were anesthetized via isoflurane and bilateral medial collateral ligament (MCL) injuries were induced via a 1 cm skin incision over the medial aspect at both the left and right stifles. The subcutaneous tissue was dissected to expose the sartorius muscle and underlying MCL, and the mid-point of the MCL was completely transected, leaving the knee capsule intact. The muscular layer was partially sutured with 5-0 Vicryl sutures, creating a pouch into which 1×10⁹ or 5×10⁹ exosomes were administered.

### Normal Dose (1×10⁹) Extracellular Vesicle Administration

Exosomes (1×10⁹) were administered into the pouch immediately following MCL injury, and the muscle layer was closed. PBS was administered into the contralateral MCL injury as a control. Subcutaneous and subdermal tissue layers were closed with 5-0 Vicryl sutures.

### High Dose (5×10⁹) Extracellular Vesicle Administration

Normal and preconditioned exosomes (5×10⁹) were administered into the pouch immediately following MCL injury in both legs and the muscle layer was closed. Subcutaneous and subdermal tissue layers were closed with 5-0 Vicryl sutures. At 3 days post injury, the contralateral MCL received a second dose of 5×10⁹ exosomes. Animals in both groups were allowed unrestricted cage movement immediately after surgery. Ligaments were collected at day 14 post-injury and used for mechanical testing. Data were analyzed via Student's T-test (treatment vs control).

### Immunohistochemistry (IHC)

Longitudinal cryosections of MCL at 14 days post injury were cut at a 5 μm thickness, mounted on Colorfrost Plus microscope slides and maintained at −70° C. IHC was performed on frozen sections. Cryosections were fixed in acetone, exposed to 3% hydrogen peroxide to eliminate endogenous peroxidase activity, blocked with Rodent Block M (Biocare Medical, Pacheco, CA), and incubated with rabbit or rat primary antibodies. Primary rat monoclonal antibodies specific to mouse F4/80, CD206, CD31 (all 1:100, BioRad, Hercules, CA) were used to detect total macrophages, M2 macrophages, and endothelial cells, respectively. Rabbit polyclonal antibodies were used for type I collagen (1:800, Abcam-Serotec, Raleigh, NC) and type III collagen (1:150, Abcam-Serotec, Raleigh, NC). Lastly, rabbit monoclonal CCR7 was used to identify M1 macrophages (1:1200, Abcam-Serotec, Raleigh, NC). After primary antibody incubation, samples were exposed to Rabbit or Rat HRP Polymer (Biocare Medical, Pacheco, CA). The bound antibody complex was visualized using diaminobenzidine (DAB). Stained sections were dehydrated, cleared, cover-slipped, and viewed using light microscopy. After IHC staining, micrographs were collected using a camera-assisted microscope (Nikon Eclipse microscope, model E6000 with an Olympus camera, model DP79). Images of 3-6 sections were captured and counted per animal. Images captured for measurement of total macrophages, M1 and M2 macrophages, endothelial cells, type I collagen, and type III collagen were quantified via Image J (National Institutes of Health, NIH). Measurements were collected 1) within the granulation tissue and 2) within the entire section. Tendon cryosections were also H&E stained to observe general morphology of the healing tendon.

### Fractal Analysis

Fractal analysis was used to quantify and evaluate collagen matrix organization and measure the rate of healing and scar formation. H&E-stained sections were cropped to 4.5 inches×4.5 inches to include the transected region. Tendons were prepared and tested as described in previous publications (Chamberlain C S, et al. (2011) Quantification of collagen organization and extracellular matrix factors within the healing ligament. Microsc Microanal 17:779-787) and (Frisch et al. (2012) Quantification of collagen organization using fractal dimensions and Fourier transforms. Acta Histochemica 114 (2): 140-144), which are incorporated herein by reference.

### Mechanical Testing

Ligaments at 14-days post injury were tested to determine the influence of exosome treatment on mechanical performance. The MCL was harvested with both femoral and tibial insertion sites intact, and the surrounding tissue and insertion site excised. The femur-MCL-tibia (FMT) complex was kept hydrated (PBS). MCL length, width, and thickness were measured using digital calipers at pre-load. Width and thickness measurements were obtained at the injury site. The cross-sectional area (assumed to be an ellipse) was then estimated. The FMT complex was mounted in a mechanical testing machine, a pre-load of 0.1 N applied to the ligament and the MCL was preconditioned with cyclically loading to 2% strain for 10 cycles. Dimension measurements for the ligament were recorded at pre-load, and the ligament pulled to failure at a rate of 1% strain per second. Failure force/maximum load was recorded as the highest load prior to failure of the ligament, and failure stress/maximum stress was calculated by dividing the failure force by the initial cross-sectional area of the ligament. Strain was calculated as the increase in FMT length divided by its preloaded length. Stiffness was calculated by determining the slope of the most linear portion of the load-displacement curve. Young's modulus was calculated by the slope of the linear portion of the stress-strain curve.

### Statistical Analysis

A one-way analysis of variance (ANOVA) was used to examine treatment differences in IHC and mechanical data. Post-hoc comparisons were performed using Fisher's LSD or Tukey's method for IHC and mechanical data, respectively. Experimental data are presented as the means±S.E.M. All p-values reported are two sided. Computations and figures were performed using KaleidaGraph, version 4.03 (Synergy Software, Inc., Reading, PA). Significance was set at p<0.05.

### Results

### Immunohistochemistry (1×10⁹ Exosomes)

Ligament healing, scar size, collagen organization, and collagen production were examined at 14 days post MCL-injury. Exosome treatment reduced the size of the forming scar and improved collagen organization (FIGS. 3E-3J). Type I and type III collagen production was increased (FIGS. 3G-3J) in exosome treated animals.

### Mechanical Testing (1×10⁹ Exosomes)

A single dose of exosomes (1×10⁹) administered directly into the MCL at the time of injury improved ligament remodeling as assessed by reduced MCL scar size, improved collagen organization, and increased Type I and Type III collagen production within the ligament (FIGS. 3E-3J). However direct exosome injection had no effect on mechanical properties of the ligament or reductions in inflammation of the MCL (FIGS. 3A-3D).

### Mechanical Testing (5×10⁹ Exosomes)

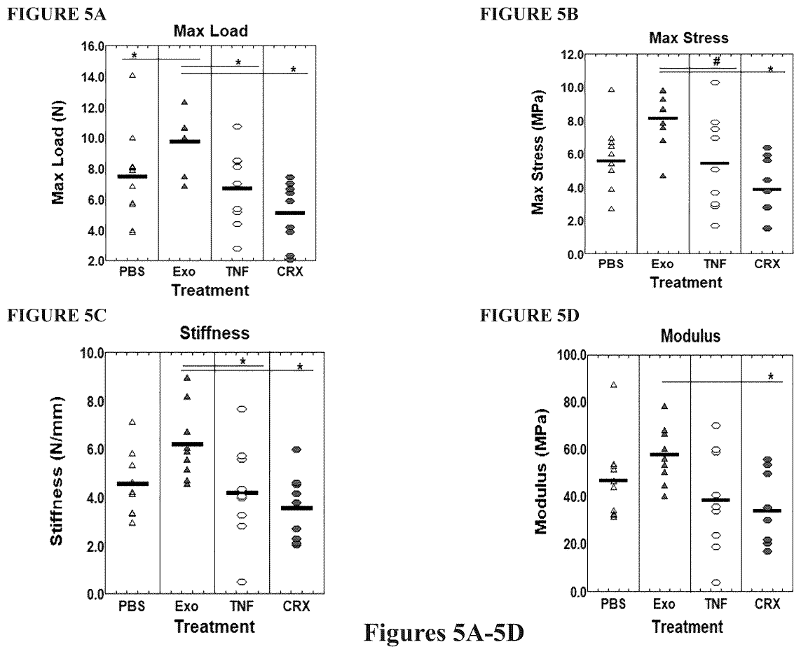

High dose exosome treatment (5×10⁹ exosomes) without preconditioning (EXO) improved max load (FIG. 5A) and max stress (FIG. 5B) compared to injured CX. Surprisingly, CRX pretreated and/or TNF pretreatment reduced stress (FIG. 5B), stiffness (FIG. 5C), and modulus (FIG. 5D) indicative of an overall reduced strength compared to the injured control. Thus, unexpectedly, whereas preconditioned exosomes did not enhance mechanical properties of the MCL, administering high-dose exosomes at the time of injury improved strength of the ligament compared to the injured control.

The inventors of the current disclosure, based on results from using 1×10⁹ exosomes, expected that the administration of preconditioned exosomes would modulate exosome behavior and improve MCL mechanical properties as preconditioned mesenchymal stem cells have been shown by the inventors to produce reparative monocytes and macrophages, thereby accelerating recovery following orthopedic damage and/or surgery. Treating monocytes or macrophages with CRX-exosomes in vitro results in alterations in gene expression, protein secretion, and cell surface markers consistent with a reduced inflammatory state, thereby promoting growth.

However, unexpectedly, TNF and CRX preconditioned exosomes had no effect on mechanical properties of the MCL (FIGS. 5A-5D). Further, surprisingly, high dose exosomes (5×10⁹ exosomes) without preconditioning resulted in improvements in MCL mechanical properties, including maximum load at 14-days post injury (FIGS. 5A-5D).

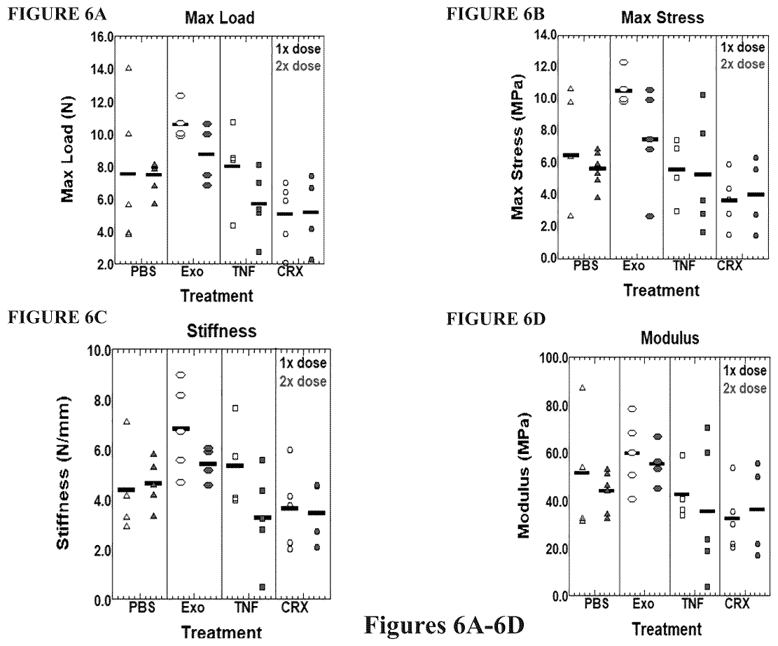

A second dose of exosomes (5×10⁹ exosomes) administered at Day 3, when macrophage infiltration is elevated, exhibited no further improvements in maximum load, maximal stress, stiffness, or modulus (FIGS. 6A-6D).

### CONCLUSION

The inventors demonstrate herein that administration of low dose exosome (1×10⁹/leg) improved ligament remodeling, but not ligament strength. In contrast, high dose exosome administration (5×10⁹/leg) improved ligament strength as demonstrated by increased maximum load. Preconditioned exosomes administration was not associated with improvements in strength and a second administration of high dose exosome (5×10⁹) administered at days 0 and day 3 had no additive benefit.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification, and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

### REFERENCES

1. Chiodo C P, Wilson M G (2006) Current concepts review: acute ruptures of the Achilles tendon. Foot & Ankle International 27:305-313.
2. de Jong J P, Nguyen J T, Sonnema A J, Nguyen E C, Amadio P C, et al. (2014) The incidence of acute traumatic tendon injuries in the hand and wrist: a 10-year population-based study. Clin Orthop Surg 6:196-202.
3. Wynn T A (2004) Fibrotic disease and the T(H)1/T(H)2 paradigm. Nature Reviews Immunology 4:583-594.
4. Xiao W, Hong H, Kawakami Y, Lowell C A, Kawakami T (2008) Regulation of myeloproliferation and M2 macrophage programming in mice by Lyn/Hck, SHIP, and Stat5. J Clin Invest 118:924-934.
5. Godwin J W, Pinto A R, Rosenthal N A (2013) Macrophages are required for adult salamander limb regeneration. Proc Natl Acad Sci USA 110:9415-9420.
6. Aktas E, Chamberlain C S, Saether E E, Duenwald-Kuehl S E, Kondratko-Mittnacht J, et al. (2016) Immune modulation with primed mesenchymal stem cells delivered via biodegradable scaffold to repair an Achilles tendon segmental defect. J Orthop Res.
7. Saether E E, Chamberlain C S, Aktas E, Leiferman E M, Brickson S L, et al. (2016) Primed Mesenchymal Stem Cells Alter and Improve Rat Medial Collateral Ligament Healing. Stem Cell Rev 12:42-53.
8. Chamberlain C S, Saether E E, Aktas E, Vanderby R (2017) Mesenchymal Stem Cell Therapy on Tendon/Ligament Healing. J Cytokine Biol 2.
9. Shen H, Kormpakis I, Havlioglu N, Linderman S W, Sakiyama-Elbert S E, et al. (2016) The effect of mesenchymal stromal cell sheets on the inflammatory stage of flexor tendon healing. Stem Cell Research & Therapy 7:144.
10. Sindrilaru A, Peters T, Wieschalka S, Baican C, Baican A, et al. (2011) An unrestrained proinflammatory M1 macrophage population induced by iron impairs wound healing in humans and mice. J Clin Invest 121:985-997.
11. Benoit M, Desnues B, Mege J L (2008) Macrophage polarization in bacterial infections. J Immunol 181:3733-3739.
12. Weisberg S P, McCann D, Desai M, Rosenbaum M, Leibel R L, et al. (2003) Obesity is associated with macrophage accumulation in adipose tissue. Journal of Clinical Investigation 112:1796-1808.
13. Kim J, Hematti P (2009) Mesenchymal stem cell-educated macrophages: a novel type of alternatively activated macrophages. Exp Hematol 37:1445-1453.
14. Bouchlaka M N, Moffitt A B, Kim J, Kink J A, Bloom D D, et al. (2017) Human Mesenchymal Stem Cell-Educated Macrophages Are a Distinct High IL-6-Producing Subset that Confer Protection in Graft-versus-Host-Disease and Radiation Injury Models. Biol Blood Marrow Transplant 23:897-905.
15. Frisch K E, Duenwald-Kuehl S E, Kobayashi H, Chamberlain C S, Lakes R S, et al. (2012) Quantification of collagen organization using fractal dimensions and Fourier transforms. Acta Histochem 114:140-144.
16. Chamberlain C S, Crowley E M, Kobayashi H, Eliceiri K W, Vanderby R (2011) Quantification of collagen organization and extracellular matrix factors within the healing ligament. Microsc Microanal 17:779-787.
17. Moestrup S K, Moller H J (2004) CD163: a regulated hemoglobin scavenger receptor with a role in the anti-inflammatory response. Ann Med 36:347-354.
18. Deng W, Chen W, Zhang Z, Huang S, Kong W, et al. (2015) Mesenchymal stem cells promote CD206 expression and phagocytic activity of macrophages through IL-6 in systemic lupus erythematosus. Clin Immunol 161:209-216.
19. Soslowsky, L. Lab website (2023) www.med.upenn.edu/orl/soslowskylab/achilles-research.html.
20. Prevalence and risk factors of a rotator cuff tear in the general population. Yamamoto A, Takagishi K, Osawa T, Yanagawa T, Nakajima D, shitara H, Kobayashi T. J Shoulder Elbow Surg. 2010 January; 19 (1): 116-20.

1. A method of treating an orthopedic injury in a subject in need thereof, the method comprising administering to the subject a population of non-preconditioned mesenchymal stromal cell (MSC)-derived extracellular vesicles thereby improving a mechanical property of the orthopedic injury.

2. The method of claim 1, wherein the orthopedic injury is selected from the group consisting of a partial tendon tear, a complete tendon tear, a partial tendon laceration, a compete tendon laceration, a partial tendon avulsion, a complete tendon avulsion, a partial ligament tear, a complete ligament tear, a partial ligament laceration, a compete ligament laceration, tendinopathy, tendinosis, tendinitis, meniscal tears, and joint capsule tears.

3. The method of claim 1, wherein the orthopedic injury is selected from the group consisting of plantar fasciitis, tennis elbow, bicep tendinitis, and carpal tunnel syndrome.

4. The method of claim 2, wherein the orthopedic injury is a medial collateral ligament (MCL) injury.

5. The method of claim 2, wherein the orthopedic injury is an anterior cruciate ligament (ACL) injury.

6. The method of claim 1, wherein the population of MSC-derived extracellular vesicles is obtained by culturing a population of bone marrow mesenchymal stem cells (BM-MSCs) in vitro and isolating the population of MSC-derived extracellular vesicles from the BM-MSCs.

7. The method of claim 1, wherein the population of MSC-derived extracellular vesicles (EVs) is administered at a dose between about 1×10⁴ EVs/kilogram and about 10×10⁹ EVs/kilogram of body weight of the subject.

8. The method of claim 1, wherein the population of MSC-derived extracellular vesicles is administered by intravenous, intramuscular, subcutaneous, or intradermal injection or infusion.

9. The method of claim 1, wherein the mechanical property is maximum load of a tendon and/or ligament.

10. The method of claim 1, wherein the mechanical property is maximum stress of a tendon and/or ligament.

11. A pharmaceutical composition, comprising a population of non-preconditioned mesenchymal stromal cell (MSC)-derived extracellular vesicles and a pharmaceutically acceptable carrier.

12. A method of improving mechanical properties of an orthopedic injury, comprising
a) administering to a subject in need thereof a population of non-preconditioned mesenchymal stromal cell (MSC)-derived extracellular vesicles to a site of an orthopedic injury, wherein the orthopedic injury comprises an injured tissue, wherein the injured tissue comprises a tendon, a ligament, cartilage, a peri-ligamentous tissue, a peri-tendinous tissue, and/or a joint capsule; and
b) improving a mechanical property of the injured tissue.

13. The method of claim 12, wherein the mechanical property is maximum load of a tendon and/or ligament.

14. The method of claim 12, wherein the mechanical property is maximum stress of a tendon and/or ligament.

15. A method of improving scar healing and/or collagen organization in a subject with an orthopedic injury, comprising:
a) preconditioning mesenchymal stromal cells (MSC);
b) isolating extracellular vesicles from the preconditioned MSCs; and
c) administering an effective amount of preconditioned MSC-derived extracellular vesicles systemically or locally to the subject.

16. The method of claim 15, wherein the population of MSC-derived extracellular vesicles is administered to the subject at a dose equal to, or less than about 1×10¹⁰ extracellular vesicles/kg of body weight.

17. A method of obtaining extracellular vesicles from a population of preconditioned mesenchymal stromal cells (MSCs), comprising the steps of:
a) obtaining a population of MSCs from bone marrow of a subject;
b) preconditioning the population of MSCs; and
c) isolating the extracellular vesicles from the population of preconditioned MSCs.

18. The method of claim 17, wherein the preconditioning comprises treating the MSCs with an inflammatory factor.

19. The method of claim 18, wherein the inflammatory factor comprises tumor necrosis factor (TNF), Interferon-gamma (IFN-γ), Interleukin-1 beta (IL-1B), Interleukin-6 (IL-6), Interleukin-17 (IL-17).

20. The method of claim 18, wherein the inflammatory factor comprises polyinosinic acid, hypoxia, lipopolysaccharide (LPS), or an LPS derivative.

21. The method of claim 17, wherein the preconditioning comprises treating the MSCs with an LPS analog.

22. The method of claim 21, wherein the LPS analog is CRX, monophosphoryl lipid A (MPLA), lipid A, Eritoran (E5564), RC-529, CRX-526, GSK1795091, and/or SMIP-30.

23. The method of claim 1, wherein the extracellular vesicles comprise one or more exosomes, microvesicles, and/or apoptotic bodies.

24. The pharmaceutical composition of claim 11, wherein the extracellular vesicles comprise one or more exosomes, microvesicles, and/or apoptotic bodies.

25. The method of claim 12, wherein the extracellular vesicles comprise one or more exosomes, microvesicles, and/or apoptotic bodies.

26. The method of claim 15, wherein the extracellular vesicles comprise one or more exosomes, microvesicles, and/or apoptotic bodies.

27. The method of claim 17, wherein the extracellular vesicles comprise one or more exosomes, microvesicles, and/or apoptotic bodies.
